(19) United States
(12) Patent Application Publication
(10) Pub. No.: US 2011/0152698 A1
(43) Pub. Date: Jun. 23, 2011

(54) METHOD AND APPARATUS FOR BLOOD PRESSURE WAVEFORM BASELINE ESTIMATION AND REMOVAL

(76) Inventors: Saul E. Greenhut, Aurora, CO (US); Mustafa Karamanoglu, Fridley, MI (US)

(21) Appl. No.: 12/646,019

(22) Filed: Dec. 23, 2009

(51) Int. Cl. A61B 5/0215

(52) U.S. Cl. 600/486

(57) ABSTRACT

An implantable medical device system including an implantable blood pressure sensor extracts a baseline signal from the sensed blood pressure signal and subtracts the extracted baseline signal from the sensed blood pressure signal to obtain a corrected pressure monitoring signal. The corrected pressure signal is monitored to detect a cardiac-related condition.

### TECHNICAL FIELD

The disclosure relates generally to implantable medical devices and, in particular, to a method and apparatus for monitoring a blood pressure signal.

### BACKGROUND

Implantable medical devices are available for monitoring physiological signals in a patient. For example, a patient's blood pressure signal may be monitored using a pressure sensor typically mounted along a transvenous lead and advanced to a desired monitoring location. A pressure sensor may be positioned within a ventricular or atrial chamber or along a vein or artery for monitoring a blood pressure signal. The blood pressure signal can be used to detect physiological events that influence the blood pressure signal or relate to the hemodynamic status of the patient. The baseline of a pressure sensor signal may wander or vary due to non-cardiac influences such as respiration, coughing, sneezing, changes in patient motion or posture, or other motion artifact. Changes in the pressure signal baseline may significantly alter pressure measurements obtained from the signal and lead to false or missed detections of actual cardiovascular-elated changes in blood pressure. Apparatus and methods are needed, therefore, for addressing baseline wander present in blood pressure signals monitored by an implantable medical device.

### BRIEF DESCRIPTION OF THE DRAWINGS

FIG. 1 is a functional block diagram of one embodiment of an implantable medical device (IMD).

FIG. 2 is a functional block diagram of a pressure signal monitoring portion of an IMD.

FIG. 3 is a flow chart of a method for monitoring a blood pressure signal according to one embodiment.

FIG. 4A is a recording of a right ventricular blood pressure signal.

FIG. 4B is a corrected blood pressure signal obtained by subtracting an extracted baseline signal from the right ventricular blood pressure signal of FIG. 4A.

FIG. 5A is a recording of a pulmonary artery pressure signal.

FIG. 5B is a recording of the corrected pulmonary artery pressure signal obtained by subtracting an extracted baseline signal from the pulmonary artery pressure signal of FIG. 5A.

### DETAILED DESCRIPTION

In the following description, references are made to illustrative embodiments. It is understood that other embodiments may be utilized without departing from the scope of the disclosure. As used herein, the term “module” refers to an application specific integrated circuit (ASIC), an electronic circuit, a processor (shared, dedicated, or group) and memory that execute one or more software or firmware programs, a combinational logic circuit, or other suitable components that provide the described functionality.

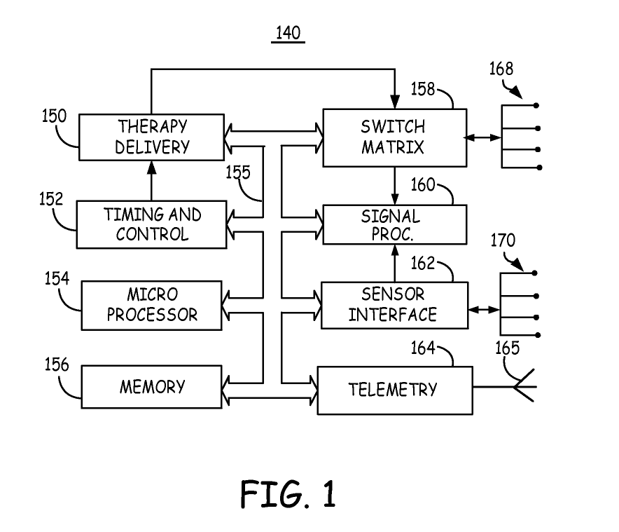

FIG. 1 is a functional block diagram of one embodiment of an implantable medical device (IMD). IMD 140 generally includes timing and control circuitry 152 and an operating system that may employ microprocessor 154 or a digital state machine for timing sensing and therapy delivery functions (when present) in accordance with a programmed operating mode. Microprocessor 154 and associated memory 156 are coupled to the various components of IMD 140 via a data/address bus 155.

IMD 140 may include therapy delivery module 150 for delivering a therapy in response to determining a need for therapy, e.g., based on sensed physiological signals. Therapy delivery module 150 may provide drug delivery therapies and/or electrical stimulation therapies. For example, therapy delivery module may include a pulse generator used to deliver cardiac pacing therapies or nerve stimulation therapies. Therapies are delivered by module 150 under the control of timing and control circuitry 152.

Therapy delivery module 150 may be coupled to two or more electrodes 168, via an optional switch matrix 158, for delivering cardiac pacing or nerve stimulation. Electrodes 168 may be carried by leads coupled to IMD 140 or incorporated on the IMD housing.

Electrodes 168 may also be used for receiving cardiac electrical signals through any unipolar or bipolar sensing configuration. Cardiac electrical signals may be monitored for use in diagnosing or managing a patient condition or may be used for determining when a therapy is needed and controlling the timing and delivery of the therapy. Signal processor 160 receives cardiac signals and includes sense amplifiers and may include other signal conditioning circuitry and an analog-to-digital converter. Cardiac electrical signals, which may be intracardiac EGM signals, far field EGM signals, or subcutaneous ECG signals, can be used to detect a need for therapy delivery and used in the timing and control of therapy. EGM/ECG signals may also be used in conjunction with the blood pressure signal for estimating a baseline pressure signal as will be described below.

IMD 140 is additionally coupled to one or more sensors 170 for sensing other physiological signals. Physiological sensors 170 include a pressure sensor and may further include other sensors, such as an activity sensor, posture sensor, or the like. Physiological sensors may be carried by leads extending from IMD 140, contained inside the IMD housing or incorporated on the IMD housing. A pressure sensor is typically provided as a transvenous blood pressure sensor advanced to a cardiovascular location to monitor pressure in a heart chamber or blood vessel. Among the pressure monitoring sites at which a blood pressure sensor is typically deployed are the right ventricle and the pulmonary artery, though numerous other locations are possible.

Sensor signals are received by a sensor interface 162 which provides sensor signals to signal processing circuitry 160. Sensor interface 162 may provide initial amplification, filtering, rectification, or other signal conditioning. Sensor signals are used by signal processor 160 and/or microprocessor 154 for detecting physiological events or conditions. In particular, signals from a pressure sensor included in sensors 170 are processed by signal processor 160 and/or microprocessor 154 for detecting hemodynamic or cardiac-related events or conditions.

The operating system includes associated memory 156 for storing operating algorithms and control parameter values that are used by microprocessor 154. The memory 156 may also be used for storing data compiled from sensed physiological signals and/or relating to device operating history for telemetry out upon receipt of a retrieval or interrogation instruction.

IMD 140 further includes telemetry circuitry 164 and antenna 165. Programming commands or data are transmitted during uplink or downlink telemetry between IMD telemetry circuitry 164 and external telemetry circuitry included in a programmer or monitoring unit.

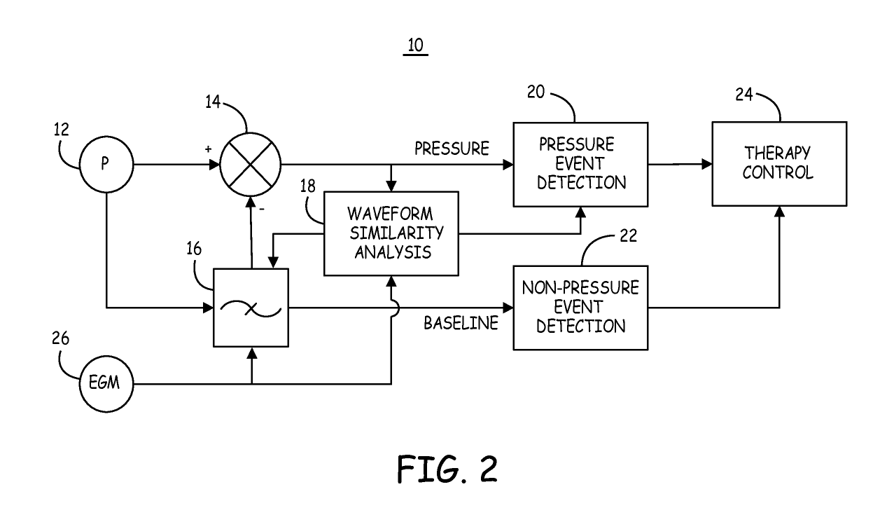

FIG. 2 is a functional block diagram 10 of a pressure signal monitoring portion of IMD 140. A “raw” pressure signal 12 is received from a blood pressure signal and provided as input to a summing block 14 and to a baseline extraction block 16. The pressure signal 12 may be low pass filtered to remove non-physiological, high frequency signal noise.

Baseline extraction block 16 extracts a time-varying baseline signal from the pressure signal 12. The baseline signal may be extracted using a number of techniques. As will be further described below, a cubic spline method may be used to estimate the baseline signal. An EGM signal 26 may optionally be provided as input to baseline extraction block 16 for establishing timing markers on each cardiac cycle for use in setting knot positions and determining baseline values between which splines are computed.

Alternatively, infinite impulse response (IIR) or finite impulse response (FIR) filters may be applied at block 16 to extract a time-varying baseline signal. In particular, an IIR Bessel filter is advantageous because of its maximally linear phase. Otherwise, it is beneficial to apply other types of IIR filters in forward and reverse directions to achieve linear (zero) phase and prevent waveform phase distortion. FIR filters inherently have linear phase and may be applied in a computationally efficient manner using multi-rate processing. Additionally, adaptive filters may be applied to automatically vary the filter characteristics as the baseline pressure frequency varies. The low-pass corner frequency of these filters should be high enough to pass respiratory and other low-frequency variations (e.g., coughing, sneezing, posture), while being low enough to reject cardiac or vascular pressure variations. This frequency is typically between approximately 0.8 and 1.0 Hz. An adaptive filter may be used to automatically vary the corner frequency.

The extracted baseline signal is subtracted from the received pressure signal 12 at summing block 14 to obtain a corrected pressure monitoring signal with baseline wander removed. The corrected pressure monitoring signal output from summing block 14 is provided as input to waveform similarity analysis block 18. EGM signal 26 may also be provided as input to waveform similarity analysis block 18 to allow the pressure waveforms to be separated beat-by-beat. The beat-to-beat pressure waveforms are compared to each other to determine if beat-to-beat variation of the pressure waveforms is within an acceptable variability threshold over a predetermined time interval, e.g. approximately 10 to 20 seconds. If the waveforms vary by more than the acceptable threshold, the beat-to-beat variation are less likely to be associated with a cardiovascular-related pressure change and may be due to baseline wander caused by non-cardiac sources.

As such, the results of the waveform similarity analysis 18 may be provided as feedback to baseline extraction block 16. If the waveform variability is within the established threshold, no adjustment is made to the baseline extraction method. If the variability is outside the acceptable limits, the baseline extraction method may be adjusted or a different method selected to improve the baseline estimation and removal from the “raw” pressure waveform. Adjustments to the baseline extraction may include adjusting the identification of knot positions during the cubic spline technique, adjusting the computation of splines, adjusting filtering parameters when other filtering methods are being used, or selecting a different baseline extraction technique.

The corrected pressure monitoring signal with baseline wander removed is provided as input to pressure event detection block 20. If the result of the waveform similarity analysis indicates non-cardiac variation in the waveforms (input from block 18), pressure event detection 20 may be suspended or certain waveforms may be rejected until the pressure waveforms meet the similarity criteria applied at block 18. Pressure event detection block 20 may perform a variety of event detection algorithms depending on the particular monitoring application. For example a right ventricular pressure signal may be monitored to derive a systolic pressure, diastolic pressure, mean pressure, estimated pulmonary artery pressure, ejection duration, a peak dP/dt, or other pressure monitoring variables used to evaluate a patient's cardiac function.

Changes in pressure variables may be used to monitor a heart failure patient, confirm or detect arrhythmias, determine a need for an IMD-delivered therapy or therapy adjustment, or for other diagnostic or therapy management purposes. Accordingly, output from pressure event detection block 20 may be provided as input to therapy control block 24. Therapy control block 24 may correspond to portions of microprocessor 152 and/or timing and control 152 in FIG. 1, used to control the delivery of therapy in response to blood pressure monitoring.

The output of baseline extraction block 16 may be provided as input to a non-pressure event detection block 22. The extracted baseline signal may contain other physiological information not directly related to cardiac function. For example, variation in the extracted baseline signal may be caused by respiration, coughs, sneezes, posture changes, motion, or the like. These types of events not directly related to cardiac hemodynamic function may be detected by non-pressure event detection block 22. While these events cause a change in the blood pressure signal, they are not used to detect blood pressure events at block 20. The non-pressure event detection, however, may be provided as input to therapy control 24 for use in making therapy delivery decisions.

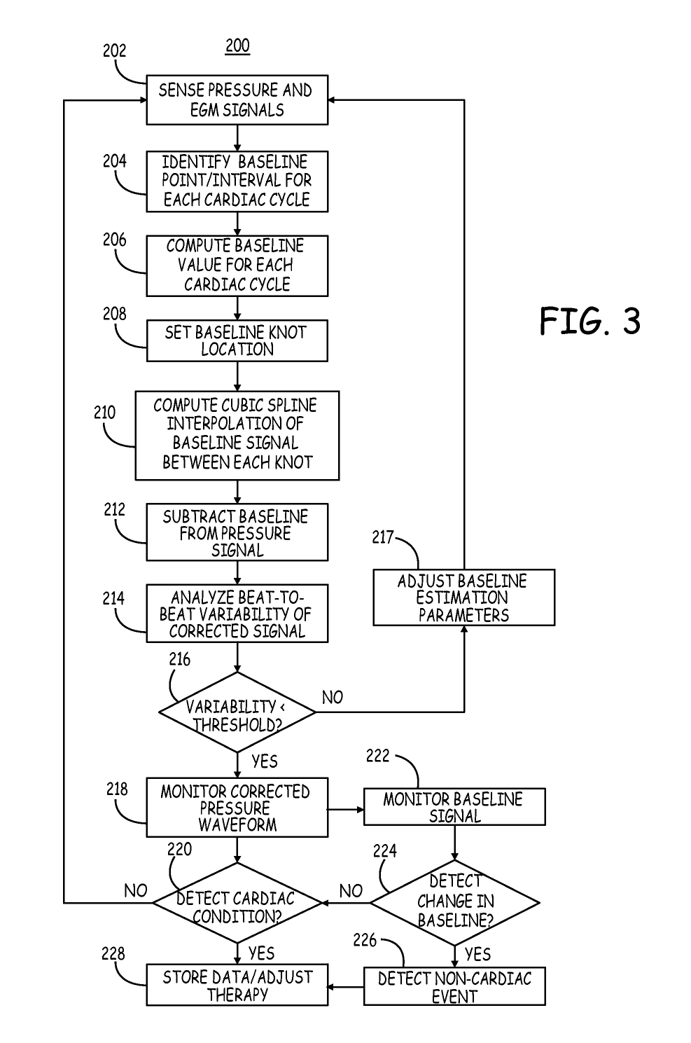

FIG. 3 is a flow chart of a method for monitoring a blood pressure signal according to one embodiment. Flow chart 200 is intended to illustrate the functional operation of the implantable medical device system, and should not be construed as reflective of a specific form of software or hardware necessary to practice the methods described. It is believed that the particular form of software will be determined primarily by the particular system architecture employed in the device system. Providing software, hardware and/or firmware to accomplish the described functionality in the context of any modern implantable medical device system, given the disclosure herein, is within the abilities of one of skill in the art.

Methods described in conjunction with flow chart 200 may be implemented in a computer-readable medium that includes instructions for causing a programmable processor to carry out the methods described. A “computer-readable medium” includes but is not limited to any volatile or non-volatile media, such as a RAM, ROM, CD-ROM, NVRAM, EEPROM, flash memory, and the like. The instructions may be implemented as one or more software modules, which may be executed by themselves or in combination with other software.

At block 202, a blood pressure signal is sensed. An EGM or ECG signal may also be sensed for used in detecting baseline points or intervals at block 204 as will be described further below. The pressure signal is expected to reach a baseline pressure associated with diastolic intervals of the cardiac cycle. The timing and duration of the baseline pressure will depend on the pressure monitoring location. For example, if an intraventricular pressure signal is being monitoring, a baseline pressure occurs prior to ventricular systole and may remain relatively flat for a short interval of time during or at the end of ventricular diastole. If the pulmonary artery pressure is being monitored, a minimum pressure may be reached just prior to the rise of systolic pressure with little or no interval of relatively flat baseline. If pressure is being monitored in an atrial chamber, the baseline will occur during atrial diastole.

A baseline point or baseline interval is identified at block 204. A baseline point may be identified using a characteristic feature of the pressure waveform or a derivative of the pressure waveform. For example, the time of the baseline point may be identified as the time of a minimum amplitude of the pressure signal, the time of a positive-going zero crossing of the first time derivative of the pressure signal (dP/dt), or the time of a maximum absolute value of the second time derivative d²P/dt². Alternatively, the EGM/ECG signal may be used to identify a baseline point on the pressure signal by identifying a pressure signal sample point occurring simultaneously with, or a selected time interval earlier than, an EGM/ECG event (e.g., an R-wave, P-wave, or a pacing pulse).

A baseline interval may be identified using features of the pressure signal, a time derivative of the pressure signal, an EGM/ECG signal, or any combination thereof. In one embodiment, an interval of relatively flat baseline is identified using the first time derivative of the pressure signal, dP/dt. The absolute values of a selected number of consecutive dP/dt sample points are summed. The resulting sum is compared to a threshold value. If the sum of N consecutive dP/dt sample points is less than the threshold, these consecutive points represent an interval of relatively flat baseline. The first sample point of the consecutive points is identified as the start of a baseline interval.

The end of the baseline interval may be identified after the start of the baseline interval as the first point of a selected number of consecutive sample points whose sum exceeds the threshold. Alternatively, the end of the baseline interval may be identified as a time point coinciding with or just prior to an EGM/ECG event, e.g., a ventricular pacing pulse or sensed R-wave.

After identifying the temporal location of the baseline point or interval, a baseline value is computed at block 206. When a single baseline point is identified, the baseline value may be determined as the amplitude of the baseline point. Alternatively, a selected number of sample points before and/or after and including the baseline point may be averaged to obtain a baseline value. If a baseline interval is identified, the baseline value may be computed using any of the baseline interval sample points. For example, the baseline value may be an average of the first N consecutive sample points identifying the start of the baseline interval and the last consecutive sample points identifying the end of the baseline interval.

At block 208, a baseline knot location is set. The knot location is typically set as the identified baseline point or the start of an identified baseline interval. Alternatively, the knot location may be set at any point along an identified baseline interval. The knot location may be set to a location along an identified baseline interval where the sample point amplitude most closely matches a computed baseline value. The knot value is set to the baseline value computed at block 206. Using this knot location and knot value as a representative baseline for each cardiac cycle, the baseline signal between consecutive knots is interpolated at block 210. The interpolated baseline may be computed using a cubic spline method as indicated in FIG. 3. Alternatively a lower-order polynomial or even a linear function may be used to interpolate the baseline signal between knots.

The computed baseline signal is subtracted from the pressure signal at block 212 to obtain a baseline-corrected pressure signal. The corrected pressure signal is also referred to herein as a “monitoring signal” because the wandering baseline signal has been removed rendering the corrected signal more reliable for monitoring blood pressure changes that are related to cardiac function without the influence of non-cardiac changes to the blood pressure signal, such as respiration, coughs, posture changes, or the like.

The corrected pressure signal may be used directly at block 218 to monitor for cardiovascular-related pressure conditions or events. In some embodiments, the beat-to-beat variability of the corrected pressure signal is analyzed first, at block 214, to verify that the baseline signal removal is optimal. The variability of the blood pressure signal over short intervals of time, for example over approximately 10 to 20 seconds, should typically be low since cardiac-related changes in blood pressure will tend to be more gradual and occur over intervals of time longer than 10 to 20 seconds. A sudden change that occurs over only one or a few cardiac cycles is typically associated with a non-cardiac cause, such as a cough, sneeze, posture change, or the like.

As described previously, if the variation of the pressure waveform from beat-to-beat within a predetermined duration of time is greater than an acceptable variability (block 216), the baseline estimation parameters may be adjusted at block 217. The pressure signal features selected to identify a baseline point or baseline interval may be changed, the method used to compute the baseline value may be adjusted, or the method used to interpolate the baseline signal may be adjusted. Alternatively, a different method for computing and removing a wandering baseline signal may be selected using one of a variety of possible IIR or FIR filter types as described above to obtain the baseline signal to increase the beat-to-beat similarity of the corrected pressure waveform.

The analysis of beat-to-beat variability performed at blocks 214 and 216 may be performed upon initial implant of the medical device to identify the best method for deriving the baseline signal in a particular patient and implant location. The variability analysis may optionally be repeated at a selected frequency to promote optimal baseline signal identification and removal from the raw pressure signal for ongoing reliable pressure monitoring.

The baseline-corrected pressure signal is monitored at block 218 to detect cardiac conditions at block 220. The detection methods used will vary depending on the particular monitoring application and on the therapy delivery capabilities of the implanted device. The detected conditions are stored at block 228 in device memory for later download telemetry and review by a clinician. Detected conditions may be used to adjust a therapy delivered by the implanted device. For example, if the device is capable of delivering a heart failure therapy such as cardiac resynchronization therapy (CRT), a ventricular pressure signal may be monitored to determine systolic pressure, diastolic pressure, mean pressure, stroke volume, cardiac output, estimated pulmonary artery diastolic pressure, or other monitored hemodynamic parameters. If a hemodynamic parameter worsens, the therapy may be adjusted accordingly. Numerous examples of pressure signal monitoring applications exist. Various examples of pressure signal monitoring applications and the use of a blood pressure signal in the control a delivered therapy are generally described in commonly-assigned U.S. Pat. No. 6,438,408 (Mulligan), U.S. Pat. No. 7,548,784 (Chinchoy), U.S. Pat. No. 7,488,291 (Cho), U.S. Pat. No. 7,367,951 (Bennett) and U.S. Pat. No. 7,181,283 (Hettrick), all of which patents are hereby incorporated by reference herein in their entirety.

At block 222, the baseline signal may be monitored to detect non-cardiac events. If a significant change in the baseline signal occurs within a relatively short interval of time at block 224, a non-cardiac event may be detected at block 226. For example, if the baseline signal changes by more than a predetermined percentage within less than approximately 10 seconds, an event such as a cough, sneeze, posture change or other non-cardiac event may be detected. The time course and magnitude of the baseline change may be useful in discriminating between different types of non-cardiac events, such as a respiratory event like a cough or a sneeze and posture change event like lying down or standing up. Notation of the non-cardiac event may be stored in memory at block 228 to provide a clinician with additional data that may be useful in interpreting physiological signal data and in diagnosing a patient condition.

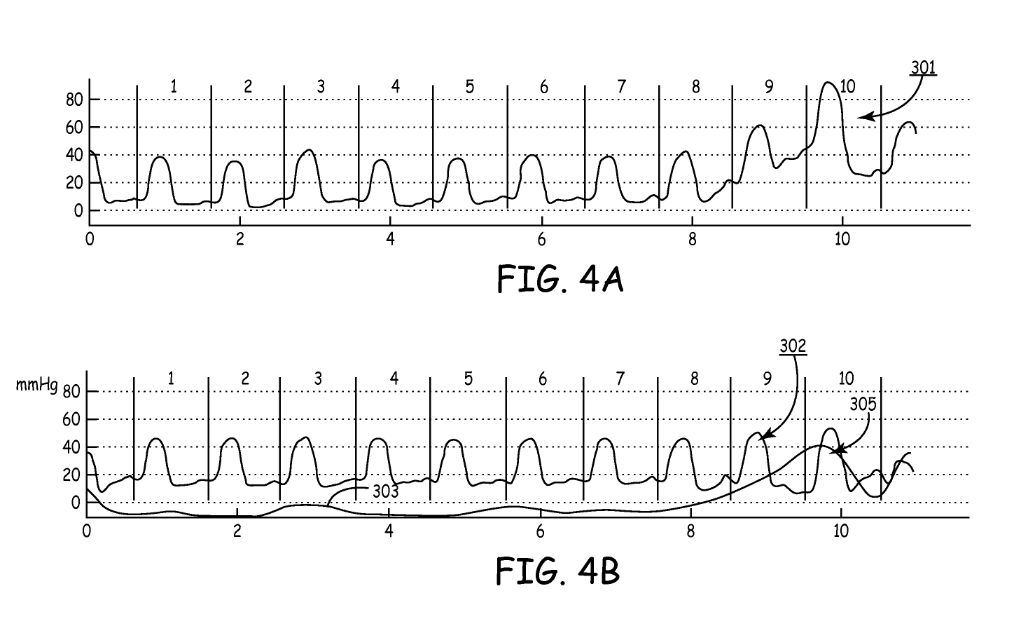

FIG. 4A is a recording of a right ventricular blood pressure signal 301. FIG. 4B is the corrected blood pressure signal 302 obtained by subtracting an extracted baseline signal 303 from the right ventricular blood pressure signal 301. In this example, the baseline signal 303 is obtained using a Chebyshev filter. As compared to the original signal 301, the beat-to-beat similarity of the corrected pressure signal 302 is increased. The removal of the wandering baseline signal promotes increased sensitivity and specificity in detecting cardiac-related pressure events or conditions.

A large increase 305 in the baseline signal 303 is seen to occur between approximately 8 and 10 seconds. The large increase 305 occurring in less than approximately 2 seconds may correspond to a cough and may be detected and identified as a non-cardiac event during pressure signal monitoring. Detection of such non-cardiac events may be used for diagnostic purposes and may be used in rejecting pressure waveforms occurring during the non-cardiac events when computing pressure monitoring parameters. Detection and storage of the frequency of non-cardiac events such as coughs may be reflective of respiratory status or an overall status of the patient and be valuable to a clinician managing the patient.

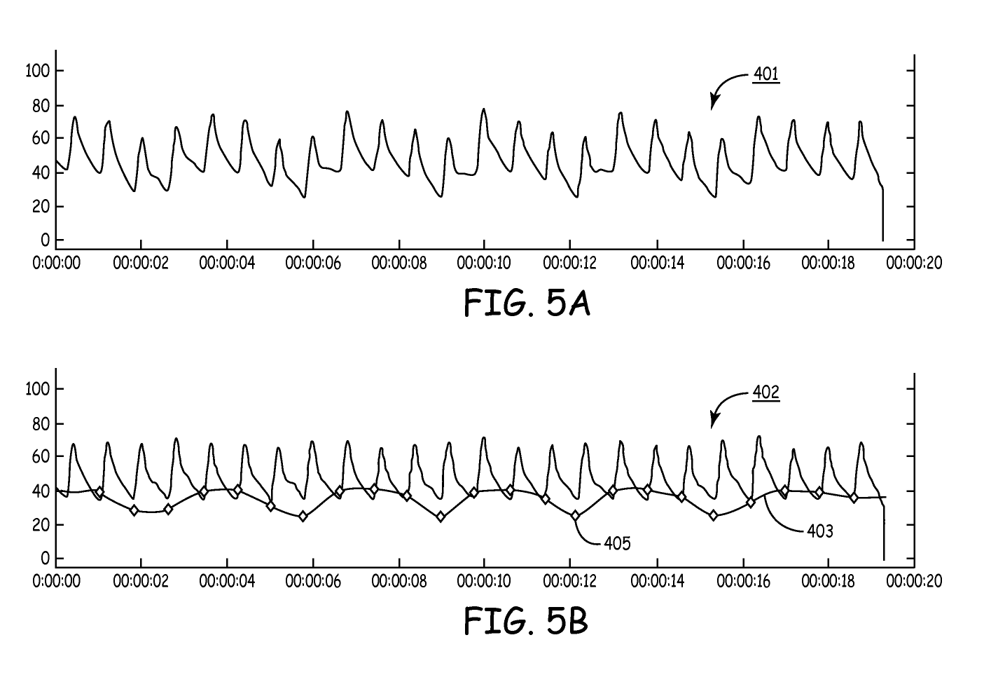

FIG. 5A is a recording of a pulmonary artery pressure signal 401. FIG. 5B is a recording of the corrected pulmonary artery pressure signal 402 obtained by subtracting an extracted baseline signal 403 from the pulmonary artery pressure signal 401. In this example, the baseline signal 403 was obtained using the cubic spline method of interpolating the baseline signal between selected knots 405, indicated by diamond markers, on each cardiac cycle. Knot locations 405 may be based on the timing of R-wave detection. In this example, cyclical baseline variation is caused by respiration and removal of this cyclical variation promotes more reliable blood pressure signal monitoring. Reduced beat-to-beat variation of the corrected pressure signal 402 is observed as compared to the uncorrected signal 401.

It is understood that while FIGS. 4A, 4B, 5A and 5B represent pressure recordings in the right ventricle and in the pulmonary artery, respectively, the methods described herein may be applied to pressure recordings obtained at other cardiovascular locations as well.

Thus, an implantable medical device system and associated method for monitoring a pressure signal have been presented in the foregoing description with reference to specific embodiments. It is appreciated that various modifications to the referenced embodiments may be made without departing from the scope of the disclosure as set forth in the following claims.

1. A method for removing baseline wander from a blood pressure signal acquired by an implantable medical device, the method comprising:
sensing a blood pressure signal using an implantable pressure sensor;
extracting a baseline signal from the sensed blood pressure signal;
subtracting the extracted baseline signal from the sensed blood pressure signal to obtain a corrected pressure monitoring signal; and
detecting a cardiac-related condition in response to the corrected pressure monitoring signal.

2. The method of claim 1 further comprising:
computing a variability of a plurality of pressure waveforms from the corrected pressure monitoring signal;
comparing the variability to an allowable threshold of variability; and
adjusting the baseline signal extraction if the measured variability exceeds the allowable threshold.

3. The method of claim 1 further comprising detecting a non-cardiac related condition in response to the extracted baseline signal.

4. The method of claim 3 further comprising storing the detected cardiac-related and non-cardiac related conditions.

5. The method of claim 4 further comprising adjusting a therapy based on the detected cardiac-related and non-cardiac related conditions.

6. The method of claim 1 wherein extracting the baseline signal comprises:
identifying a baseline point on the pressure signal for each of a plurality of cardiac cycles;
computing a baseline value of the pressure signal for each of the identified baseline points; and
interpolating the baseline signal between the identified baseline points using the computed baseline values.

7. The method of claim 6 wherein identifying a baseline point comprises:
computing the first time derivative of the pressure signal;
summing a plurality of consecutive sample points of the first time derivative;
comparing the sum of the plurality of consecutive sample points to a threshold;
selecting a sample point of the sensed pressure signal corresponding in time to one of the plurality of consecutive sample points as the baseline point in response to the sum being less than the threshold.

8. The method of claim 7 wherein computing the baseline value comprises using a sample point of the sensed pressure signal corresponding in time to at least one of the plurality of consecutive sample points to compute the baseline value.

9. The method of claim 6 wherein identifying the baseline point comprises:
detecting a cardiac electrical event;
selecting a sample point of the sensed pressure signal corresponding to the detected cardiac electrical event.

10. The method of claim 6 wherein computing the baseline value comprises:
detecting a cardiac electrical event;
selecting a plurality of sample points of the sensed pressure signal corresponding to the cardiac electrical event;
computing the baseline value using at least one of the plurality of sample points of the sensed pressure signal correlated in time to the cardiac electrical event.

11. An implantable medical device system, comprising:
an implantable blood pressure sensor; and
a processor coupled to the pressure sensor and configured to:
receive a pressure signal from the pressure sensor;
extract a baseline signal from the sensed blood pressure signal;
subtract the extracted baseline signal from the sensed blood pressure signal to obtain a corrected pressure monitoring signal; and
detect a cardiac-related condition in response to the corrected pressure monitoring signal.

12. The system of claim 11 wherein the processor is further configured to:
compute a variability of a plurality of pressure waveforms of the corrected pressure monitoring signal;
compare the variability to an allowable threshold of variability; and
adjust the baseline signal extraction if the measured variability exceeds the allowable threshold.

13. The system of claim 11 wherein the processor is further configured to detect a non-cardiac related condition in response to the baseline signal.

14. The system of claim 13 further comprising a memory for storing the detected cardiac-related and non-cardiac related conditions.

15. The system of claim 14 further comprising a therapy delivery module, the processor configured to adjust a therapy delivered by the therapy delivery module based on the detected cardiac-related and non-cardiac related conditions.

16. The system of claim 11 wherein extracting the baseline signal comprises:
identifying a baseline point on the pressure signal for each of a plurality of cardiac cycles;
computing a baseline value of the pressure signal for each of the identified baseline points; and
interpolating the baseline signal between the identified baseline points using the computed baseline values.

17. The system of claim 16 wherein identifying a baseline point comprises:
computing the first time derivative of the pressure signal;
summing a plurality of consecutive sample points of the first time derivative;
comparing the sum of the plurality of consecutive sample points to a threshold;
selecting a sample point of the sensed pressure signal corresponding in time to one of the plurality of consecutive sample points as the baseline point in response to the sum being less than the threshold.

18. The system of claim 17 wherein computing the baseline value comprises using a sample point of the sensed pressure signal corresponding in time to at least one of the plurality of consecutive sample points to compute the baseline value.

19. The system of claim 16 further comprising electrodes for sensing cardiac electrical signals and delivering cardiac pacing pulses,
the processor coupled to the electrodes and receiving cardiac electrical event signals;
the processor is configured to identify the baseline point by selecting a sample point of the sensed pressure signal correlated in time to a cardiac electrical event.

20. The system of claim 16 further comprising electrodes for sensing cardiac electrical signals and delivering cardiac pacing pulses,
the processor coupled to the electrodes and receiving cardiac electrical event signals;
the processor is configured to select a plurality of sample points of the sensed pressure signal correlated in time to a cardiac electrical event and compute the baseline value using at least one of the plurality of sample points of the sensed pressure signal corresponding to the cardiac electrical event.

21. A computer-readable medium storing a set of instructions which when implemented in a processor of an implantable medical device system comprising an implantable blood pressure sensor cause the system to:
sense a blood pressure signal using the implantable pressure sensor;
extract a baseline signal from the sensed blood pressure signal;
subtract the extracted baseline signal from the sensed blood pressure signal to obtain a corrected pressure monitoring signal; and
detect a cardiac-related condition in response to the corrected pressure monitoring signal.
